(19) United States
(12) Patent Application Publication
(10) Pub. No.: US 2005/0112729 A1
(43) Pub. Date: May 26, 2005

(54) Recombinant DNA having hydantoinase gene and carbamylase gene and process for producing amino acid

(75) Inventors: Ikuo Kira, Kanagawa (JP); Yasuhiro Takenaka, Kanagawa (JP); Hiroyuki Nozaki, Kanagawa (JP); Kunihiko Watanabe, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/961,116

(22) Filed: Oct. 12, 2004

Related U.S. Application Data
(63) Continuation of application No. PCT/JP03/04553, filed on Apr. 10, 2003.

(30) Foreign Application Priority Data
Apr. 10, 2002 (JP) 2002-108550

(51) Int. Cl. C12P 13/04; C12N 9/80; C12N 1/21

(52) U.S. Cl. 435/106; 435/228; 435/252.33

(57) ABSTRACT

The object of the present invention is to prepare a recombinant DNA efficiently co-expressing genes of enzymes such as HHase, and to produce an amino acid efficiently from hydantoin by utilizing such a recombinant DNA. The present invention provides a recombinant DNA having base sequences encoding the following gene regions (I) and (II) incorporated therein in a predetermined direction and in a predetermined order to produce an amino acid efficiently: (I) a gene region containing a base sequence encoding a hydantoinase gene and a trp promoter sequence located upstream of the base sequence encoding the hydantoinase gene regulating expression of the hydantoinase gene, and (II) a gene region containing a base sequence encoding a carbamylase gene and a trp promoter sequence located upstream of the base sequence encoding the carbamylase gene regulating expression of the carbamylase gene.

### TECHNICAL FIELD

The present invention relates to a recombinant DNA and a process for producing an amino acid by using the same, and in particular to a recombinant DNA co-expressing a hydantoinase gene and a carbamylase gene efficiently and a process for producing an amino acid with high productivity by using the same.

### BACKGROUND ART

One of known methods for producing an amino acid by use of an enzyme is asymmetric decomposition of a 5-substituted hydantoin compound. The 5-substituted hydantoin compound may be synthesized chemically at a low cost as the starting material, and may be decomposed to be an optically active amino acid. This method for producing the optically active amino acids from the 5-substituted hydantoin compounds is important in the production of pharmaceutical preparations, chemical products, food additives, etc.

As one of methods for producing the optically active amino acid from the 5-substituted hydantoin compound, there is a method using the following enzymes (A) and (B):
(A) An enzyme that catalyzes hydrolytic reaction of the 5-substituted hydantoin compound to form an N-carbamylamino acid; this enzyme is called hydantoinase (also known as hydantoin hydrolase; the enzyme may be referred to hereinafter as “HHase”).
(B) An enzyme that catalyzes hydrolytic reaction of the obtained N-carbamylamino acid to form an amino acid; this enzyme is called carbamylase (also known as N-carbamylamino acid hydrolase; the enzyme may be referred to hereinafter as “CHase”).

If at least one of the enzymes (A) HHase and (B) CHase is optically selective, an optically active amino acid may be produced from the 5-substituted hydantoin compound.

Use of the aforementioned enzymes may be performed by introducing genes encoding HHase and CHase into a host such as Escherichia coli to prepare a transformant, and then culturing this transformant, whereby an amino acid may be formed from the 5-substituted hydantoin and accumulated in the medium. A method for producing an amino acid by utilizing a recombinant DNA is reported in, for example, WO 01/23582 and WO 05/8449. In the techniques described therein, a rhamnose promoter is used as a promoter for expression of HHase, etc.

### DISCLOSURE OF THE INVENTION

There have been developed a variety of practical techniques as to ligation of an extraneous gene with a recombinant vector and introduction of such a vector into a cell such as a microorganism for the expression of the genes in the cell. Examples of the techniques directed to amino acid production may include those described in the above two literatures. The techniques described in the literatures utilize a rhamnose promoter. It is necessary to add rhamnose as an inducer at a predetermined time for exerting the function of the rhamnose promoter. Upon adding rhamnose, it is very important to adjust the time for addition. Industrial production of an amino acid using such techniques thus requires an additional step for adding the inducer, the timing of which has to be carefully regulated. Therefore, the production steps thereof are undesirably complicated.

Lac promoter is one of the well-known promoters. In the industrial mass production of an amino acid with a transformed microorganism, the microorganisms should be maintained at a high density. In order to maintain such a high-density in the culture, glucose may have to be added as a nutrient source. However, addition of glucose leads to suppression of the lac promoter, and thus use of the lac promoter may not be preferable in the industrial production of an amino acid.

Accordingly, it is necessary to chose suitable combination of the promoters and host, etc. depending on specific situations. What is possible at the laboratory level is not always industrially advantageous. Employment of a promoter and the like that are known to be capable of enhancing expression level does not always result in satisfactory production. Further, when a plurality of enzymes are to be expressed as described above, the co-expression of the genes should be performed in a well-balanced manner to meet the final objectives.

The present invention has been made in the light of the aforementioned viewpoints. The object of the present invention is to provide a recombinant DNA that is capable of efficiently co-expressing genes of enzymes such as HHase, etc. and to efficiently produce an amino acid from hydantoin by utilizing such a recombinant DNA.

The present inventors have made extensive studies for obtaining a recombinant DNA capable of efficiently co-expressing HHase and CHase genes. As a result, they have found out that, among a large number of promoters, a trp promoter is capable of efficiently expressing HHase, CHase and hydantoin racemase (which may be referred to hereinafter as “HRase”) and suitable in the industrial production of the amino acid. The present inventors have also found out that the activity of each enzyme is significantly affected by the arrangement of the HHase and CHase genes upon introducing them into the recombinant DNA, such as the direction of the genes in a recombinant DNA and the order of insertion of the genes, thereby completing the present invention.

That is, the present invention is as follows:
[1] A recombinant DNA comprising a base sequence encoding the following gene region (I), and another base sequence encoding the following gene region (II):

(I) a gene region containing a base sequence encoding a hydantoinase gene, and a trp promoter sequence located upstream of the base sequence encoding the hydantoinase gene, said trp promoter regulating expression of the hydantoinase gene;

(II) a gene region containing a base sequence encoding a carbamylase gene, and a trp promoter sequence located upstream of the base sequence encoding the carbamylase gene, said trp promoter regulating expression of the carbamylase gene.
[2] The recombinant DNA according to the above-mentioned [1], wherein the transcriptional direction of the base sequence encoding the gene region (I) is the same as that of the base sequence encoding the gene region (II), and wherein the base sequence encoding the gene region (I) and the base sequence encoding the gene region (II) are disposed in this order along the transcriptional direction.
[3] The recombinant DNA according to the above-mentioned [1], wherein the transcriptional direction of the base sequence encoding the gene region (I) is in the reverse direction of the base sequence encoding the gene region (II).
[4] The recombinant DNA according to the above-mentioned [1], wherein the transcriptional direction of the base sequence encoding the gene region (I) is the same as that of the base sequence encoding the gene region (II), and wherein the base sequence encoding the gene region (II) and the base sequence encoding the gene region (I) are disposed in this order along the transcriptional direction.
[5] The recombinant DNA according to any one of the above-mentioned [1] to [4], wherein the recombinant DNA is a plasmid.
[6] A transformed cell transformed with the recombinant DNA according to any one of the above-mentioned [1] to [5].
[7] A transformed cell transformed with:

the recombinant DNA according to any one of the above-mentioned [1] to [5]; and

a recombinant DNA having a base sequence encoding a hydantoin racemase gene and a trp promoter sequence located upstream of the hydantoin racemase gene, said trp promoter regulating expression of the hydantoin racemase gene.
[8] The transformed cell according to the above-mentioned [7], wherein the recombinant DNA according to any one of the above-mentioned [1] to [5] is a low-copy recombinant DNA, and the recombinant DNA having the base sequence encoding the hydantoin racemase gene and the trp promoter sequence located upstream of the hydantoin racemase gene for regulating expression of the hydantoin racemase gene is a high-copy recombinant DNA.
[9] The transformed cell according to any one of the above-mentioned [6] to [8], wherein the host of the recombinant DNA is Escherichia coli.
[10] A process for producing a mixture of proteins comprising the step of culturing the transformed cell according to the above-mentioned [6] in a medium, to accumulate a protein having a hydantoinase activity and a protein having a carbamylase activity in the medium and/or the transformed cell.
[11] A process for producing a mixture of proteins comprising the step of culturing the transformed cell according to the above-mentioned [7] in a medium, to accumulate a protein having a hydantoinase activity, a protein having a carbamylase activity and a protein having a hydantoin racemase activity in the medium and/or the transformed cell.
[12] A process for producing an amino acid comprising the step of culturing the transformed cell according to any one of the above-mentioned [6] to [9] in a medium to give a culture, and the step of mixing 5-substituted hydantoin with the culture to form the amino acid.

### BRIEF DESCRIPTION OF THE DRAWINGS

FIG. 1 is a diagram showing the location and direction of inserted HHase and CHase in a plasmid.

FIG. 2 is a diagram showing trp promoter cassettes 1 and 2.

FIG. 3 is a diagram showing the construction of pTrpHfCf.

FIG. 4 is a diagram showing the construction of pTrpCrHf.

FIG. 5 is a diagram showing the construction of pTrpHrCr.

FIG. 6 is a diagram showing the construction of pTrp4R.

FIG. 7 is a diagram showing the construction of pTrp8CH.

### BEST MODE FOR CARRYING OUT THE INVENTION

The present invention will be described hereinbelow in detail with an order of “1. Recombinant DNA, etc.”, “2. Process for producing the enzyme protein”, and “3. Process for producing an amino acid.”

1. Recombinant DNA, etc.

The recombinant DNA of the present invention contains base sequences encoding the following gene regions (I) and (II):

(I) A gene region containing a base sequence encoding an HHase gene and a trp promoter sequence located upstream of the HHase gene sequence for regulating expression of the HHase gene (this gene region may be referred to hereinafter as “trp+HHase”. In the Drawings, the trp promoter is referred to as “Ptrp”.)

(II) A gene region containing a base sequence encoding a CHase gene and a trp promoter sequence located upstream of the CHase gene sequence for regulating expression of the CHase gene (this gene region may be referred to hereinafter as “trp+CHase”).

That is, the recombinant DNA of the present invention has the trp promoter as a promoter for expressing each of HHase and CHase. The trp promoter is found in e.g., Escherichia coli and located upstream of genes belonging to a gene group encoding some enzymes involved in biosynthesis of tryptophan. In the present invention, a known trp promoter may be used. The trp promoter is commercially available in the form of a trp promoter-containing cloning vector or a cell containing such a vector. Employment of the trp promoter results in a sufficient expression of HHase and CHase. The trp promoter does not require addition of an inducer such as rhamnose that is requisite when a rhamnose promoter is employed. Accordingly, steps and treatments accompanying addition of the inducer can be obviated in the present process for producing the amino acid. Further, the activity of the trp promoter is not suppressed by glucose, and thus the expression levels of the enzymes are not suppressed even if glucose is used in a high-density culture of the transformed microorganisms in the production of the amino acid on an industrial scale.

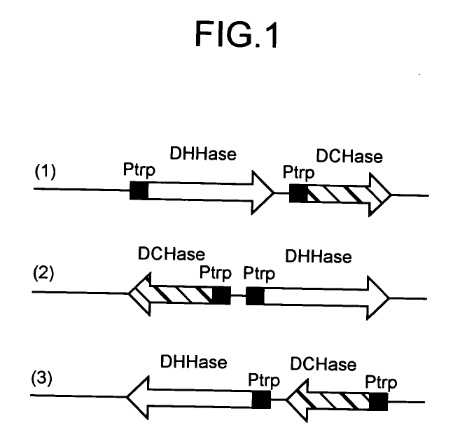

There are three embodiments in terms of the directions and order of insertion of the DNA having the base sequence encoding HHase (also referred to hereinafter as “DNA encoding HHase”) and the DNA having the base sequence encoding CHase (also referred to hereinafter as “DNA encoding CHase”), into a recombinant DNA such as a vector.
(First embodiment) The base sequence encoding the gene region (I) and the base sequence encoding the gene region (II) are inserted in the same transcriptional direction, and in the order of the base sequence encoding the gene region (I) and then the base sequence encoding the gene region (II), along the transcriptional direction (FIG. 1(1)). That is, in FIG. 1(1), the transcriptional directions of both the “trp+HHase” gene region and “trp+CHase” gene region are from left to right; and the “trp+HHase” gene region and “trp+CHase” gene region are disposed in this order from left to right. In FIG. 1, DHHase and DCHase that are specific for D-isomer substrates are employed as the examples of HHase and CHase, respectively.
(Second embodiment) The base sequence encoding the gene region (I) and the base sequence encoding the gene region (II) are inserted such that the transcriptional directions thereof are opposed from each other (FIG. 1(2)). That is, in FIG. 1(2), the transcriptional direction of the “trp+CHase” gene region is from right to left, while the transcriptional direction of the “trp+HHase” gene region is from left to right.
(Third embodiment) The base sequence encoding the gene region (I) and the base sequence encoding the gene region (II) are inserted in the same transcriptional direction, and in the order of the base sequence encoding the gene region (II) and then the base sequence encoding the gene region (I), along the transcriptional direction (FIG. 1(3)). That is, the transcriptional directions of both the “trp+HHase” gene region and “trp+CHase” gene region are from right to left, and the “trp+CHase” gene region and “trp+HHase” gene region are disposed in this order from right to left.

In the examples shown in FIG. 1, “Ptrp” refers to a trp promoter. “DHHase” is HHase having a D-hydantoinase activity, and “DCHase” is CHase having a D-carbamylase activity. Each arrow means that transcription proceeds in the direction of that arrow.

Among these three embodiments, the third embodiment (FIG. 1(3)) results in the highest co-expression of HHase and CHase. That is, the third embodiment may achieve an efficient, the best-balanced co-expression of the two sorts of the enzyme genes incorporated into one recombinant. The third embodiment is thus particularly preferable in the industrial production of the amino acid on a large scale.

One of possible options for expressing two or more sorts of enzymes in a transformant may be preparation of a plurality of recombinant DNAs each containing only one sort of genes to be expressed, which are then introduced into a host. However, such a transformant may result in an imbalanced expression of the genes due to difference in copy numbers in the host and disappearance of the recombinant DNA. In the present invention, however, two enzyme genes are integrated in one recombinant DNA, by which these genes may be efficiently co-expressed, resulting in the reduced possibility of the imbalance in the expression level of HHase and CHase due to the division and proliferation of the host.

In the production of the amino acids with HHase and CHase, it is desirable that both the enzymes are expressed at high levels. However, in terms of the comparison between HHase and CHase in the reaction system, a relatively higher activity of CHase is desired for efficient production of the amino acid. When the two genes encoding HHase and CHase are integrated in one recombinant, it is desirable that their expression occurs with a suitable balance for the industrial production of the amino acid. The third embodiment is the best in this respect.

The DNAs encoding HHase and CHase, which are to be integrated in the recombinant DNA of the present invention, may be those known in the art. HHase may be a protein having a hydantoinase activity, and CHase may be a protein having a carbamylase activity. An optically active amino acid, i.e. either L-amino acid or D-amino acid, may be selectively produced by employing an optically selective enzyme or enzymes as one or more of HHase and CHase. The optical selectivity of the enzyme means that the enzyme specifically uses either L- or D-amino acid as the substrate upon catalyzing the reaction.

HHase for the optically selective hydrolysis of the 5-substituted hydantoin compound may be obtained in accordance with the following manner. As an example of DHHase for the production of N-carbamyl-D-amino acid, it is known that a thermostable enzyme having such an activity exists in microorganisms of genus Bacillus. For example, HHase or an HHase-containing fraction may be prepared from Bacillus stearothermophilus ATCC 31195, etc. (Appl. Microbiol. Biotechnol. Vol.43 PR 270,1995). ATCC 31195 is available from American Type Culture Collection (Address: 12301, Parklawn Drive, Rockville, Md. 20852, United States of America). It is also known that LHHase specific to L-isomers of the hydantoin compound is present in e.g. Bacillus sp. AJ 12299 (Japanese Patent Application Laid-open No. S63-24894 Publication). Bacillus sp. AJ 12299 is a microorganism deposited on Jul. 5, 1986 under FERM-P8837 with the National Institute of Bioscience and Human-Technology, the Agency of Industrial Science of Technology, the Ministry of International Trade and Industry, JP and transferred on Jun. 27, 2001 as FERM BP-7646 under the Budapest Treaty to International Patent Organism Depositary (IPOD), National Institute of Advanced Industrial Science and Technology (AIST) (Chuo No. 6, Higashi 1-1-1, Tsukuba City, Ibaraki Pref., JP).

It is known that HHase having no optical selectivity is present in e.g. Arthrobacter aurescens, as well as Microbacterium liquefaciens AJ 3912 (J. Biotechnol. 61,1 (1998)).

It is known that CHase specifically hydrolyzing the D-isomer of the N-carbamylamino acid is present in, e.g. Pseudomonas sp. AJ 11220 (Japanese Patent Application No. S56-003034 Publication). As a result of re-identification, it has been revealed that Pseudomonas sp. AJ 11220 in fact belongs to Agrobacterium sp. Agrobacterium sp. AJ 11220 is a microorganism deposited on Dec. 20, 1977, as FERM-P4347 with the National Institute of Bioscience and Human-Technology, the Agency of Industrial Science of Technology, the Ministry of International Trade and Industry, JP, and transferred on Jun. 27, 2001 as FERM BP-7645 under the Budapest Treaty to International Patent Organism Depositary (IPOD), National Institute of Advanced Industrial Science and Technology (AIST). It is known that CHase selectively hydrolyzing the L-isomer of N-carbamylamino acid is present in, e.g. Flavobacterium sp. AJ 3912 (Japanese Patent Application No. S56-008749 Publication) and Bacillus sp. AJ 12299. As mentioned above, Flavobacterium sp. AJ 3912 is now classified into Microbacterium liquefaciens AJ 3912 (FERM-P3133), and the microorganism was deposited on Jun. 27, 1975, as FERM-P3133 with the National Institute of Bioscience and Human-Technology, the Agency of Industrial Science of Technology, the Ministry of International Trade and Industry, JP, and transferred on Jun. 27, 2001 as FERM BP-7643 under the Budapest Treaty to International Patent Organism Depositary (IPOD), National Institute of Advanced Industrial Science and Technology (AIST). Bacillus sp. AJ 12299 was deposited on Jul. 5, 1986, as FERM-P8837 with the National Institute of Bioscience and Human-Technology, the Agency of Industrial Science of Technology, the Ministry of International Trade and Industry, JP, and transferred on Jun. 27, 2001 as FERM BP-7646 under the Budapest Treaty to International Patent Organism Depositary (IPOD), National Institute of Advanced Industrial Science and Technology (AIST).

In the production of the amino acid by the recombinant DNA of the present invention, examples of the preferable combinations of the DNAs encoding HHase and CHase to be incorporated into the recombinant DNA may include any combination of HHase and CHase each selected from the following. The combination selected from the following will result in the selective production of a D-amino acid.

The preferable DNA encoding HHase may be selected from the following (i) to (iv):
(i) a DNA having the base sequence of SEQ ID NO:1 in the Sequence Listing,
(ii) a DNA having a base sequence that hybridizes under stringent conditions with a DNA consisting of a base sequence complementary to the base sequence of SEQ ID NO:1 in the Sequence Listing,
(iii) a DNA encoding an amino acid sequence of SEQ ID NO:2 in the Sequence Listing, and
(iv) a DNA encoding an amino acid sequence wherein in the amino acid sequence of SEQ ID NO:2 in the Sequence Listing, one or several amino acid residues are replaced, deleted, inserted, added or inverted.

The preferable DNA encoding CHase may be selected from the following (v) to (viii):
(v) a DNA having the base sequence of SEQ ID NO:3 in the Sequence Listing,
(vi) a DNA having a base sequence that hybridizes under stringent conditions with a DNA consisting of a base sequence complementary to the base sequence of SEQ ID NO:3 in the Sequence Listing,
(vii) a DNA encoding an amino acid sequence of SEQ ID NO:4 in the Sequence Listing, and
(viii) a DNA encoding an amino acid sequence wherein in the amino acid sequence of SEQ ID NO:4 in the Sequence Listing, one or several amino acid residues are replaced, deleted, inserted, added or inverted.

As used herein, the “stringent conditions” refer to those conditions under which a specific hybrid is formed whereas an unspecific hybrid is not formed. Although it is difficult to precisely define these conditions with numerals, examples thereof may be the conditions under which DNA molecules having higher homology, e.g. preferably not less than 50 percent, more preferably not less than 80 percent, still more preferably not less than 90 percent homology, hybridize with each other while DNA molecules having lower homology do not hybridize with each other, and the conditions under which hybridization occurs under usual washing conditions in Southern hybridization, that is, at a salt concentration corresponding to 1×SSC and 0.1 percent SDS at 60° C., preferably 0.1×SSC and 0.1 percent SDS at 60° C., more preferably 0.1×SSC and 0.1 percent SDS at 65° C.

The term “several” means a number which results in no substantial deterioration of the steric structure of a protein of amino acid residues or the activity of DHHase or DCHase, and is typically 2 to 50, preferably 2 to 30, and more preferably 2 to 10.

The combination of DHHase selected from the aforementioned (i) to (iv) and DCHase selected from the aforementioned (v) to (viii) is particularly advantageous for achieving a necessary enzyme activity for the industrial production of the D-amino acid on a large scale.

The DNAs in SEQ ID NOS:1 to 3 in the Sequence Listing were isolated from chromosomal DNA from Flavobacterium sp. AJ 11199 (FERM-P4229) (Japanese Patent Application No. S56-025119 Publication). Flavobacterium sp. AJ 11199 (FERM-P4229) was initially deposited as Alcaligenes aquamarinus with the National Institute of Bioscience and Human-Technology, the Agency of Industrial Science of Technology, the Ministry of International Trade and Industry, JP, but as a result of re-identification, it was revealed that the microorganism should have been classified into Flavobacterium sp. Flavobacterium sp. AJ 11199 is a microorganism which was deposited on Sep. 29, 1977, as FERM-P4229 with the National Institute of Bioscience and Human-Technology, the Agency of Industrial Science of Technology, the Ministry of International Trade and Industry, JP, and transferred on May 30, 2002 as FERM BP-8063 under the Budapest Treaty to International Patent Organism Depositary (IPOD), National Institute of Advanced Industrial Science and Technology (AIST).

The recombinant DNA carrying the genes encoding HHase, CHase and HRase is preferably a vector for introducing these genes into host cells. Examples of such vectors may include pHSG series (Takara Shuzo), pUC series (Takara Shuzo), pPROK series (Clontech), pSTV series (Takara Shuzo), pTWV series (Takara Shuzo), pKK233-2 (Clontech), a pBR322 series plasmid, and derivatives thereof. The “derivatives” are those obtained by modifying the plasmid by replacement, deletion, insertion, addition or inversion of a nucleotide. As referred to herein, the modification may include mutation treatment with a mutagen or UV irradiation, and modification by natural mutation. Depending on the type of the origin of replication, the copy number of the plasmids and derivatives thereof may vary in host cells. Examples of the high-copy plasmids may include pHSG series, pUC series and pPROK series plasmids, whereas examples of the low-copy plasmids may include pSTV series, pTWV series, pKK233 series and pBR322 series plasmids.

For increasing the amount of the protein production, it is preferable to connect a terminator, i.e. a transcription termination sequence, to a downstream region of the protein gene. Examples of the terminators may include rmB terminator, T7 terminator, fd phage terminator, T4 terminator, a terminator for tetracycline resistance gene, a terminator for Escherichia coli trpA gene, etc. A terminator such as rmB terminator is preferable in terms of improving the stability of the plasmid as well.

For facilitating selection of the transformant, the vector may preferably have a marker such as an ampicillin resistance gene and a kanamycin resistance gene.

A DNA fragment having the promoter, the DNA encoding HHase, the DNA encoding CHase, and the terminator each disposed therein at a predetermined location may be ligated to a vector DNA to give a recombinant DNA.

The recombinant DNA obtained in the aforementioned manner may be introduced into a host cell to give a transformant. In the production of the protein on a large scale with the recombinant DNA technology, the host cells to be transformed may be microbial cells, Actinomyces cells, yeast cells, fungal cells, plant cells, animal cells, etc. Because there are a lot of findings on production of proteins on a large scale by use of enterobacterium, it is generally preferable to use enterobacterium. In particular, Escherichia coli JM109, particularly (DE3) strain is preferable.

As another example of the process for producing an amino acid according to the present invention, HRase may be used together with HHase and CHase, as will be described in detail. In this case, HRase may be produced by the transformant as well, and then utilized in the production of the amino acid. That is, a recombinant DNA having a DNA encoding HRase may be prepared and then introduced into a host cell for transformation.

The DNA encoding HRase for use in the present invention may be those known in the art. Preferable examples thereof may be as follows:
(ix) a DNA having the base sequence of SEQ ID NO:5 in the Sequence Listing,
(x) a DNA having a base sequence that hybridizes under stringent conditions with a DNA having a base sequence complementary to the base sequence of SEQ ID NO:5 in the Sequence Listing,
(xi) a DNA encoding an amino acid sequence of SEQ ID NO:6 in the Sequence Listing, and
(xii) a DNA encoding an amino acid sequence wherein in the amino acid sequence of SEQ ID NO:6 in the Sequence Listing, one or several amino acid residues are replaced, deleted, inserted, added or inverted.
   The terms “stringent conditions” and “several” herein have the same meaning as defined in the explanation of DNAs encoding HHase, etc.

The DNA of SEQ ID NO:5 in the Sequence Listing was isolated and purified from Microbacterium liquefaciens AJ 3912. Microbacterium liquefaciens AJ 3912 was initially deposited as Flavobacterium sp. AJ 3912 (FERM-P3133) on Jun. 27, 1975 with the National Institute of Bioscience and Human-Technology, the Agency of Industrial Science of Technology, the Ministry of International Trade and Industry, Japan, but it was revealed as a result of re-identification that this microorganism should have been classified into Aureobacterium liquefaciens. Due to change of the name of this species, Aureobacterium liquefaciens has been reclassified into Microbacterium liquefaciens, and the microorganism has been deposited as Microbacterium liquefaciens AJ 3912 (National Deposition No. FERM-P3133 and International Deposition No. FERM BP-7643) with International Patent Organism Depositary (IPOD), National Institute of Advanced Industrial Science and Technology (AIST), Japan.

In the recombinant DNA carrying the DNA encoding HRase, it is preferable that the expression of the DNA encoding HRase is also regulated by the trc promoter. The terminator, the basic recombinant DNA, the selection marker and the host for the recombinant DNA having HRase gene integrated therein may preferably be the same as those described in the recombinant DNA carrying HHase gene, etc.

The recombinant DNA having the DNA encoding HRase may be introduced for expression into a host cell into which the DNA encoding HHase and the DNA encoding CHase have also been introduced, whereby the three enzymes for producing amino acids may be expressed by using one transformant, which makes the operation easier than in production using a plurality of transformants. In this case, the recombinant DNA having the DNA encoding HRase is preferably prepared separately from the recombinant DNA encoding HHase, etc. In consideration of the size of the enzyme gene and other factors, construction of a recombinant DNA by integrating the three enzyme genes into one recombinant DNA may not be practical. Accordingly, a transformant into which two sorts of recombinant DNAs were introduced is preferably prepared in order to express HRase as well.

Generally, simultaneous expression of two or more sorts of high-copy recombinant DNAs in one host cell is difficult. It is thus preferable that, when two or more sorts of recombinant DNAs are to be introduced, one of the recombinant DNAs is a high-copy recombinant DNA, and the other is a relatively low-copy recombinant DNA. The amount of the enzyme produced by each DNA, the stability of the plasmid, etc. may be taken into consideration for determining which recombinant DNA are incorporated into the high-copy recombinant DNA. In the production of the amino acid with the recombinant DNA of the present invention, it is preferable to incorporate the recombinant DNA having HRase into a high-copy recombinant DNA, and the recombinant DNA having HHase and CHase into a low-copy recombinant DNA. Whether the DNA is high-copy or not may be judged by preparing transformants having each of the recombinant DNAs and measuring the activity of the enzyme produced by each transformant.

Procedures for handling plasmids, DNA fragments, various enzymes, preparation of transform ants, and screening of the transformants in the present invention may be carried out in accordance with known techniques described in Molecular Cloning, A Laboratory Manual, 2nd Edition, edited by J. Sambrook et al., 1989, Cold Spring Harbor Laboratory Press, etc.

2. Process for producing the enzyme protein

HHase and CHase may be expressed and produced in a large amount by culturing cells that have been transformed with the recombinant DNA as described above. When the DNA encoding HRase has also been introduced, HRase may also be expressed and produced. As the medium for the production, media that are usually used in the cultivation of E. coli, for example M9-casamino acid medium, LB medium, etc. may be used. Specific conditions for carrying out the cultivation and inducing the production may suitably be selected depending on the types of the marker in the employed vector, the host microorganism, etc.

After the cultured transformant cells are collected by centrifugation, etc., the transformant cells may be disrupted or lyzed. HHase and/or CHase may then be recovered therefrom, which may be used as a crude enzyme solution. The disruption may by performed by the methods such as ultrasonic disruption, French press disruption, glass bead disruption, etc. Lysis of the microorganism may be performed by treatment with egg white lysozyme or a peptidase, or any suitable combination thereof. If necessary, these enzymes may be purified before use. The purification may be performed by conventional techniques such as precipitation, filtration and column chromatography. A purification method with antibodies to the enzymes may also be used.

In a preferable example for the production of the protein on a large scale by recombinant DNA technology, the protein may be aggregated in the transformant producing the protein to constitute a protein inclusion body. The advantage of this production method lies in protection of the desired protein against digestion with proteases present in the microorganism as well as in easy purification of the desired protein by disruption and centrifugation of the microorganism.

The protein inclusion body thus obtained may be solubilized by denaturation of the protein. After the restoration of the activity by e.g., removing the denaturant, the inclusion body may be converted into a correctly folded, physiologically active protein. There are many examples of such conversion, e.g. restoration of the activity of human interleukin-2 (Japanese Patent Application No. S61-257931 Publication).

Retrieval of the active protein from the protein inclusion body may require a series of procedures such as solubilization and activity restoration, etc., which may make the procedures more complex than in direct production of the active protein. However, if the protein to be produced in the cells in a large amount may affect growth of the microorganism, accumulation of the protein in the form of such an inert inclusion body in the cells may contribute to suppress influence of such a protein on the microorganism.

Production of the objective protein as the inclusion body on a large scale may be performed by sole expression of the desired protein under the control of a strong promoter, as well as expression of a fused protein consisting of the objective protein and another protein that is known to be expressed in a large amount.

It may be useful to dispose a sequence that is recognizable by a restriction protease in a suitable position for cutting the objective protein out after the expression of the fused protein.

The protein inclusion body thus formed as the fused protein may be recovered and then solubilized with a denaturant. The inclusion body may be solubilized together with microbial proteins. However considering the subsequent purification procedures, it is preferable to take the inclusion body out before solubilization. The inclusion body may be recovered from the microorganism by a method known in the art. For example, the microorganism may be disrupted and the inclusion body may then be recovered by treatments such as centrifugation. Examples of the denaturant for solubilizing the protein inclusion body may include guanidine hydrochloride (e.g., 6 M, pH 5 to 8) and urea (e.g. 8 M).

The enzyme protein may be regenerated as an active protein by dialysis for removing the denaturant. The dialysis solution used in dialysis may be Tris-HCl buffer or phosphate buffer, and the concentration thereof may be 20 mM to 0.5 M, and the pH thereof may be 5 to 8.

The concentration of the protein in the regeneration step is preferably regulated to be no grater than about 500 μg/ml. In order to prevent self-crosslinkage of the regenerated enzyme protein, the dialysis temperature is preferably 5° C. or less. In addition to this dialysis method, examples of the method for removing the denaturant may further include a dilution method and an ultrafiltration method, with any of which the activity may be restored.

3. Process for producing an amino acid

In the process for producing the amino acid according to the present invention, the amino acid may be produced by culturing in a medium a transformed cell that has been obtained by introducing the recombinant DNA encoding enzymes such as HHase as described above to give a cultured product containing the enzymes such as HHase formed therein, and then mixing the cultured product with the 5-substituted hydantoin to form an amino acid. The cultured product may contain HHase and CHase if the DNA encoding HHase and CHase has been introduced into the transformed cell. The resulting cultured product may also contain HRase if the DNA encoding HRase has also been introduced. Insofar as these predetermined enzymes are contained, the form of the cultured product is not particularly limited. That is, the 5-substituted hydantoin may be added directly to a medium containing the transformant, or the 5-substituted hydantoin may be mixed with the transformant such as the microorganism separated from a medium, a washed transformant, a treated material obtained by disrupting or lyzing the transformant, a crude enzyme solution containing recovered HHase, etc., or a purified enzyme solution, whereby the desired amino acid can be produced.

The reaction steps for forming an amino acid from a 5-substitued hydantoin with HHase and CHase is shown in the following reaction scheme (I):

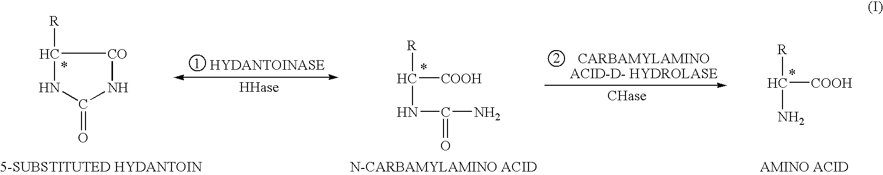

If it is desired to produce an optically active amino acid, HHase having optical selectivity may be used to form either N-carbamyl-L-amino acid or N-carbamyl-D-amino acid. The carbamylamino acid may then be subjected to the reaction with CHase, to produce the optically active amino acid.

Even if HHase has no optical selectivity in hydrolysis activity, employment of CHase having an optical selectivity will result in the production of the optically active D- or L-amino acid. In this case, it may be predicted that the unreacted one of the N-carbamylamino acid enantiomers would remain in the reaction liquid; that is, if CHase selectively decomposes N-carbamyl-L-amino acid to produce the L-amino acid, N-carbamyl-D-amino acid would remain in the reaction liquid, while if CHase selectively produces D-amino acid, N-carbamyl-L-amino acid would remain. In this case, however, HHase can also slightly catalyze the reverse reaction of dehydrating and condensing the remaining unreacted enantiomer of N-carbamylamino acid to form the 5-substituted hydantoin compound again, and therefore the optically active amino acid may be produced with high yield (at least 50 percent molar yield) by the two enzymes, that is, HHase and highly optically selective CHase or by a material containing such two enzymes.

Subsequently, as an example of the reaction for forming an optically active amino acid from DL-5-substituted hydantoin, the reaction for optically selective production of D-amino acid by HRase is shown in the reaction scheme (II):

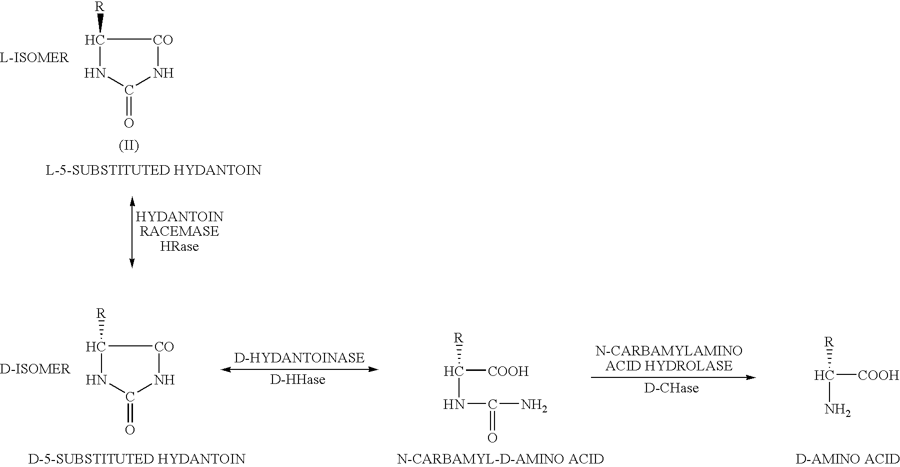

Since HRase in the protein mixture catalyzes the racemization of the 5-substituted hydantoin compound as shown in the reaction scheme (II), employment of HRase theoretically enables production of the D-amino acid with a molar yield of 100 percent from the DL-5-substituted hydantoin compound.

The process for producing the amino acid of the present invention employs the 5-substituted hydantoin compounds as the substrate for HHase or HRase. Examples of the 5-substituted hydantoin compounds may include 5-substituted hydantoin compounds corresponding to the natural amino acids such as hydantoin, 5-methyl hydantoin, 5-benzyl hydantoin, 5-(4-hydroxybenzyl)hydantoin, 5-indolylmethyl hydantoin, 5-(3,4-dihydroxybenzyl)hydantoin, 5-(p-hydroxybenzyl)hydantoin, 5-(3′-pyridyl)-methyl hydantoin, 5-methyl thioethyl hydantoin, 5-isopropyl hydantoin, 5-isobutyl hydantoin, 5-sec-butyl hydantoin, 5-carboxyethyl hydantoin, 5-carboxymethyl hydantoin, 5-(4-aminobutyl)hydantoin, and 5-hydroxymethyl hydantoin, as well as 5-substituted hydantoins corresponding to artificial amino acids such as 5-phenyl hydantoin, 5-(4-hydroxyphenyl)hydantoin, 5-methoxymethyl hydantoin, 5-benzyloxymethyl hydantoin, and 5-(3,4-methylenedioxybenzyl)hydantoin, and dihydrouracil, or derivatives thereof.

The enzyme proteins may also be used to produce N-carbamylamino acid. For example, it is possible to produce an N-carbamylamino acid by adding an L- or D-CHase inhibitor to the mixture of proteins to terminate the hydrolysis reaction at the stage of N-carbamylamino acid.

When a cultured liquid of cells, separated cells, washed cells, or treated cells that have been transformed with the recombinant DNA, or a crude enzyme solution or a purified enzyme obtained from the treated microbial material are used for catalyzing the amino acid-forming reaction, a reaction liquid containing the 5-substituted hydantoin compound and the cultured liquid, the separated cells, the washed cells, the treated cells, the crude enzyme solution, or the purified enzyme may be left stand or stirred for 8 hours to 5 days in a suitable temperature in a range of 25 to 40° C. while the pH is kept at 5 to 9.

When a water-soluble medium is employed for the cultivation of the cells that have been transformed with the recombinant DNA and the simultaneous amino acid-forming reaction, the water-soluble medium therefor may contain the 5-substituted hydantoin compound and nutrients such as a carbon source, a nitrogen source, and inorganic ions necessary for growth of the transformed cells. Further, addition of organic micronutrients such as vitamins, amino acids, etc. leads to desirable results in most of cases. An amount of the 5-substituted hydantoin compound to be added may be divided in portions and added separately. Culturing may preferably be conducted for 8 hours to 5 days under aerobic conditions while maintaining suitable pH and temperature in ranges of pH 5 to 9 and 25 to 40° C., respectively.

The amount of the D-amino acid in the cultured liquid or reaction liquid may be rapidly determined by a method known in the art. Simple measurement may be thin layer chromatography with, e.g. HPTLC CHIR manufactured by Merck. Measurement with higher analytical accuracy may be performed by high performance liquid chromatography (HPLC) with an optical resolution column such as CHIRALPAK WH manufactured by Daicel Chemical Industries, Ltd.

The amino acid thus produced may be separated and purified by techniques known in the art. Examples of the method therefor may include adsorption of basic amino acid by contacting the same with an ion-exchange resin which is followed by elution and crystallization thereof. Alternatively, the eluent may be passed through activated charcoal for discoloration and then crystallized.

Although it depends on the conditions for culturing the transformant such as Escherichia coli, employment of the recombinant DNA of the present invention using the trp promoter may achieve productivity (amount of amino acids per unit time) of approximately five times as the productivity achieved with other promoters such as rhamnose promoter.

### EXAMPLES

The present invention is further described in detail with reference to Examples 1 to 8. However, the present invention is not limited thereto.

### Example 1

### Expression of D-hydantoinase gene derived from AJ 11199 strain in E coli

1. Construction of an expression plasmid

1-1. Construction of a plasmid carrying a trp promoter

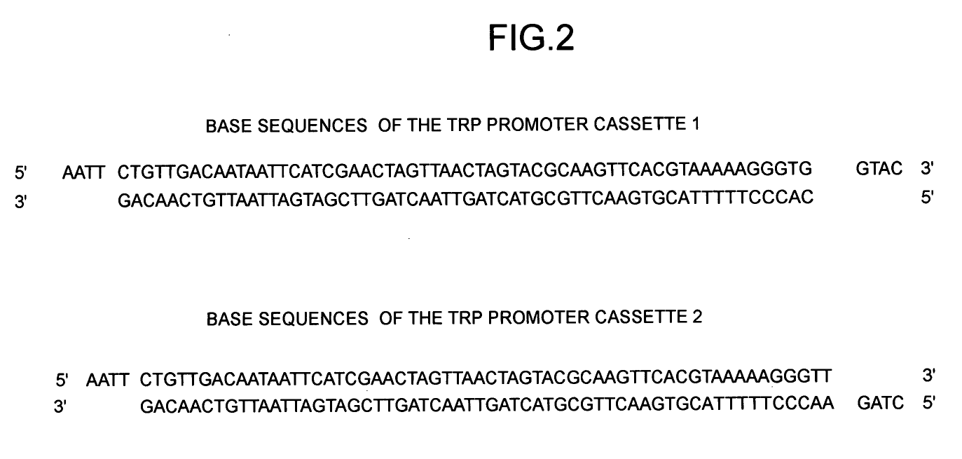

pHSG298 (manufactured by Takara Shuzo Co., Ltd.) was treated with EcoRI/KpnI, and the resulting DNA fragment was ligated to a trp promoter cassette 1 (FIG. 2). The solution of this ligation product was used to transform E. coli JM109. Among the resulting kanamycin-resistant strains, a strain having the objective plasmid was selected, and this plasmid was designated pTrp298EK. In the Sequence Listing, SEQ ID NO:21 describes the upper chain of the trp promoter cassette 1 shown in FIG. 2.

1-2. Construction of a plasmid carrying a D-hydantoinase gene

The objective gene was amplified by PCR with a chromosomal DNA from Flavobacterium sp. AJ 11199 as a template and the oligonucleotides shown in Table 1 as primers (four combinations, wherein one of (1) to (4) was combined with (5)). These fragments were treated with KpnI/XbaI, and the resulting DNA fragments were ligated to pTrp298EK that had previously been treated with KpnI/XbaI. The solution containing this ligation product was used to transform E. coli JM109. Among the resulting kanamycin-resistant strains, strains having the objective plasmids were selected. The plasmids carrying the DNA fragment that had been amplified with one of the primers (1) to (4) and the primer (5) were designated pTrp298DHHase1, 2, 3, and 4, respectively.

TABLE 1

Primers used in amplification of the
D-hydantoinase gene derived from AJ11199

| | | | |
|---|---|---|---|
| \mathring{1} | 5′-side | GGGGTACCGGAGCTGATACCATGACC | SEQ ID NO:7 |
| | | KpnI | |
| \mathring{2} | 5′-side | GGGGTACCGCAACCGATAGCCGGAGC | SEQ ID NO:8 |
| | | KpnI | |
| \mathring{3} | 5′-side | GGGGTACCATGACCCATTACGATCTC | SEQ ID NO:9 |
| | | KpnI | |
| \mathring{4} | 5′-side | GGGGTACCACCATGACCCATTACGAT | SEQ ID NO:10 |
| | | KpnI | |
| \mathring{5} | 3′-side | GCTCTAGACGTCCTGTCCTTTCCGCC | SEQ ID NO:11 |
| | | XbaI | |

2. Preparation of a cell-free extract

E. coli JM109 having pTrp298DHHase1, 2, 3, or 4 was cultured at 30° C. for 16 hours in LB medium (2 ml) containing 100 μg/ml kanamycin. 100 μl of the cultured liquid thus obtained was transferred to 4 ml of medium I (1 percent casamino acid, 1 percent peptone, 1 percent glucose, 0.5 percent ammonium sulfate, 0.1 percent monopotassium phosphate, 0.3 percent dipotassium phosphate, 0.05 percent magnesium sulfate, 0.01 percent kanamycin (pH 7.0)), and cultured at 37° C. for 16 hours. After the cultivation, the microorganism was collected from 1 ml of the cultured liquid by centrifugation (10,000 g×10 minutes), and the resulting microorganism was washed with 100 mM potassium phosphate buffer (KPB, pH 7.5). This washed microorganism was suspended in 0.5 ml of the same buffer and disrupted by ultrasonic disruption (20 kHz, 15 minutes, with UCW-201 manufactured by Tohsho Denki). The liquid obtained by the ultrasonic disruption was centrifuged (10,000 g×10 minutes) to give a supernatant as a cell-free extract.

3. Confirmation of D-hydantoinase gene expression by SDS polyacrylamide gel electrophoresis

10 μl of the cell-free extract was applied to an SDS polyacrylamide gel (Multigel 10/20 manufactured by Daiichi Pure Chemicals Co., Ltd.) and electrophoresed in accordance with the method of Laemelli et al. As a result, a band of a protein corresponding to D-hydantoinase was recognized only in the cell-free extract of the transformant having pTrp298DHHase3.

4. Measurement of D-hydantoinase activity

The E. coli JM109 having pTrp298DHHase3 was cultured at 30° C. for 16 hours in LB medium (2 ml) containing 100 μg/ml kanamycin. 0.5 ml of the cultured liquid thus obtained was transferred to 50 ml of medium II (1 percent casamino acid, 1 percent peptone, 0.2 percent glucose, 0.5 percent ammonium sulfate, 0.1 percent monopotassium phosphate, 0.3 percent dipotassium phosphate, 0.05 percent magnesium sulfate, 0.01 percent manganese sulfate, 0.01 percent kanamycin (pH 7.0)) and cultured at 37° C. for 24 hours.

After the cultivation, the microorganism was collected from 5 ml of the cultured liquid by centrifugation (10,000 g×10 minutes) and washed with 100 mM KPB (pH 7.5). The microorganism thus washed was suspended in 5 ml of the same buffer, and 0.1 ml of this suspension was added to 0.9 ml of a substrate solution (133 mg/dl D-5-benzylhydantoin, 1.1 mM MnSO₄, 100 mM KPB (pH 7.5)) and reacted at 37° C. for 60 minutes. After the reaction, 0.9 ml of a solution for terminating the reaction (1.1 mM CuSO₄) was added to 0.1 ml of the reaction liquid and then centrifuged, and the resulting supernatant was subjected to HPLC (column: CHIRALPAK WH, 0.46×25 cm, eluting solution: 1 mM CuSO₄, flow rate: 1.5 ml/min., temperature: 50° C., detection wavelength: 210 nm) to quantify the formed N-carbamyl-D-Phe. One unit of the enzyme was defined as the amount of the enzyme that produces 1 μmol of N-carbamyl-D-Phe per minute under these conditions.

As a result, the cultured liquid of the E. coli JM109 having pTrp298DHHase3 exhibited 1.5 units per mL of D-hydantoinase activity.

### Example 2

### Expression of D-carbamylase gene derived from AJ 11199 strain in E. coli

1. Construction of a plasmid carrying a D-carbamylase gene

The objective gene was amplified by PCR with a chromosomal DNA from Flavobacterium sp. AJ 11199 as a template and the oligonucleotides shown in Table 2 as primers (two combinations wherein either (1) or (2) was combined with (3)). These fragments were treated with KpnI/XbaI, and the resulting DNA fragments were ligated to pTrp298EK that had previously been treated with KpnI/XbaI. The solution containing this ligation product was used to transform E. coli JM109. Among the resulting kanamycin-resistant strains, strains having the objective plasmids were selected. The plasmids carrying a fragment that had been amplified with one of the primers (1) or (2) and the primer (3) were designated pTrp298DCHase1 and 2, respectively.

Then, pTrp298DCHase1 was treated with EcoRI/XbaI to give a DNA fragment having the trp promoter and the carbamylase gene ligated thereto, and this DNA fragment was ligated to pHSG299 (Takara Shuzo) that had previously been treated with EcoRI/XbaI. The solution containing this ligation product was used to transform E. coli JM109. Among the resulting kanamycin-resistant strains, a strain having the objective plasmid was selected, and the plasmid therein was designated pTrp299DCHase1.

TABLE 2

Primers used in amplification of the
D-carbamylase gene derived from AJ11199

| | | | |
|---|---|---|---|
| \mathring{1} | 5′-side | GGGGTACCGGAGAGGAATATGCCAGG | SEQ ID |
| | | KpnI | NO:12 |
| \mathring{2} | 5′-side | GGGGTACCGGGAAAGCAAAAGGAGAGG | SEQ ID |
| | | KpnI | NO:13 |
| \mathring{3} | 3′-side | GCTCTAGAGCCGCGCCGCTCAGACGG | SEQ ID |
| | | XbaI | NO:14 |

2. Preparation of a cell-free extract

Preparation of a cell-free extract of the E. coli JM109 having pTrp298DCHase1, 2, or pTrp299DCHase1 was conducted in the same manner as in the preparation of the cell-free extract described in Example 1.

3. Confirmation of D-carbamylase gene expression by SDS polyacrylamide gel electrophoresis

10 μl of the cell-free extract was applied to an SDS polyacrylamide gel (Multigel 10/20 manufactured by Daiichi Pure Chemicals Co., Ltd.) and electrophoresed in accordance with the method of Laemelli et al. As a result, a band of a protein corresponding to D-carbamylase was recognized only in the cell-free extract of the transformant having pTrp299DCHase1.

4. Measurement of D-carbamylase activity

Preparation of cells of the E. coli JM109 having pTrp299DCHase1 was conducted in the same manner as the preparation performed in the measurement of enzyme activity described in Example 1. The washed microorganism obtained thereby was suspended in 5 ml of 100 mM KPB (pH 7.5), and 0.1 ml of this suspension was added to 0.9 ml of a substrate solution (458 mg/dl N-carbamyl-D-Phe, 100 mM KPB (pH 7.5)) and reacted at 37° C. for 60 minutes. After the reaction, 0.9 ml of the solution for terminating the reaction was added to 0.1 ml of the reaction liquid and then centrifuged, and the resulting supernatant was subjected to HPLC to quantify the formed D-Phe. One unit of the enzyme was defined as the amount of the enzyme that produces 1 μmol of D-Phe per minute under these conditions.

As a result, the cultured liquid of the E. coli JM109 having pTrp299DCHase1 exhibited 0.6 unit per mL of D-carbamylase activity.

### Example 3

### Expression of hydantoin racemase gene derived from AJ 3912 in E. coli

1. Construction of an expression plasmid

1-1. Construction of a plasmid carrying a trp promoter

pHSG298 and pHSG299 were treated with EcoRI/XbaI, and each of the resulting DNA fragments was ligated to a trp promoter cassette 2 (FIG. 2). The solution of each of these ligation products was used to transform E. coli JM 109. Among the resulting kanamycin-resistant strains, strains having the objective plasmids were selected, and these plasmids were designated pTrp298EK and pTrp299EX, respectively. In the Sequence Listing, SEQ ID NO:22 describes the upper chain of the trp promoter cassette 2 shown in FIG. 2.

1-2. Construction of a plasmid carrying a hydantoin racemase gene

The objective gene was amplified by PCR with a chromosomal DNA from Microbacterium liquefaciens AJ 3912 as a template and the oligonucleotides shown in Table 3 as primers (two combinations wherein either (1) or (2) was combined with (3)). These fragments were treated with XaI/PstI, and each of the resulting DNA fragments was ligated to each of pTrp298EK and pTrp299EX that had previously been treated with XbaI/PstI. Each solution containing each of these ligation products was used to transform E. coli JM109, and from the resulting kanamycin-resistant strains, strains having the objective plasmids were selected. The plasmids carrying a DNA fragment that had been amplified with one of the primers (1) or (2) and the primer (3) were designated pTrp298HRase1, 2, and pTrp299HRase 1, 2, respectively.

Then, pTrp299HRase2 was treated with EcoRI/PstI to give a DNA fragment having the trp promoter and the hydantoin racemase gene ligated thereto, and this DNA fragment was ligated to pSTV29 (Takara Shuzo) that had previously been treated with EcoRI/PstI. The solution containing this ligation product was used to transform E. coli JM109. Among the resulting chloramphenicol-resistant strains, a strain having the objective plasmid was selected, and the plasmid was designated pTrp29HRase.

TABLE 3

Primers used in amplification of the
hydantoinase gene derived from AJ3912

| | | | |
|---|---|---|---|
| \mathring{1} | 5′-side | GCTCTAGAATGAGAATCCATGTCATC | SEQ ID NO:15 |
| | | XbaI | |
| \mathring{2} | 5′-side | GCTCTAGAGCGATGCGTATCCATGTC | SEQ ID NO:16 |
| | | XbaI | |
| \mathring{3} | 3′-side | GGCTGCAGCGCTCCTTCTCGTTAGAG | SEQ ID NO:17 |
| | | PstI | |

2. Measurement of hydantoin racemase activity

Preparation of the E. coli JM109 having pTrp298HRase1 or 2, pTrp299HRase1 or 2, or pTrp29HRase was conducted in the same manner as the preparation performed in the measurement of enzyme activity described in Example 1. Each of the washed microorganisms obtained thereby was suspended in 5 ml of 100 mM KPB (pH 7.5), and 0.1 ml of this suspension was added to 0.9 ml of a substrate solution (133 mg/dl D-5-benzylhydantoin, 100 mM KPB (pH 7.5)) and reacted at 37° C. for 15 minutes. After the reaction, 0.9 ml of the solution for terminating reaction was added to 0.1 ml of the reaction liquid and then centrifuged, and the resulting supernatant was subjected to HPLC to quantify the formed L-5-benzylhydantoin. One unit of the enzyme was defined as the amount of the enzyme that produces 1 μmol of L-5-benzylhydantoin per minute under these conditions. The results are shown in Table 4.

TABLE 4
Hydantoinase racemase activity of the transformant having each
plasmid

| | Hydantoin racemase activity |
|---|---|
| Plasmid | (U/ml culture liquid) |
| pTrp298HRase-1 | 4.47 |
| pTrp298HRase-2 | 0.31 |
| pTrp299HRase-1 | 6.24 |
| pTrp299HRase-2 | 7.76 |
| pTrp29HRase | 0.45 |

### Example 4

### Co-expression of D-hydantoinase gene and D-carbamylase gene derived from AJ 11999 in E. coli

1. Construction of a expression plasmid

1-1. Construction of pTrpHfCf plasmid

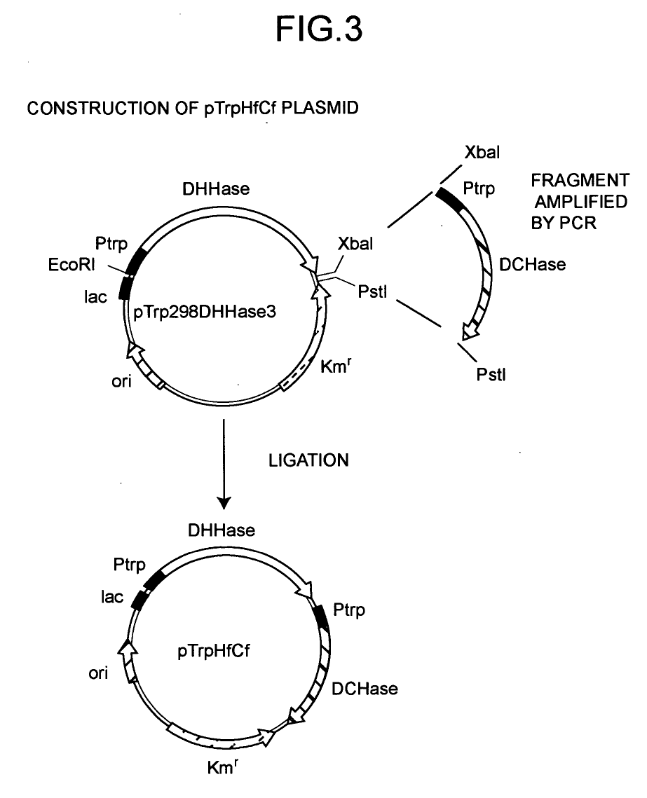

PCR was conducted with pTrp299DCHase1 as a template and the oligonucleotides (1) and (2) shown in Table 5 as primers. The resulting DNA fragment was treated with XbaI/PstI and then ligated to pTrp298DHHase3 that had previously been treated with XbaI/Pst. The solution of this ligation product was used to transform E. coli JM109. Among the resulting kanamycin-resistant strains, a strain having the objective plasmid was selected, and this plasmid was designated pTrpHfcf (FIG. 3).

1-2. Construction of pTrpCrHf plasmid

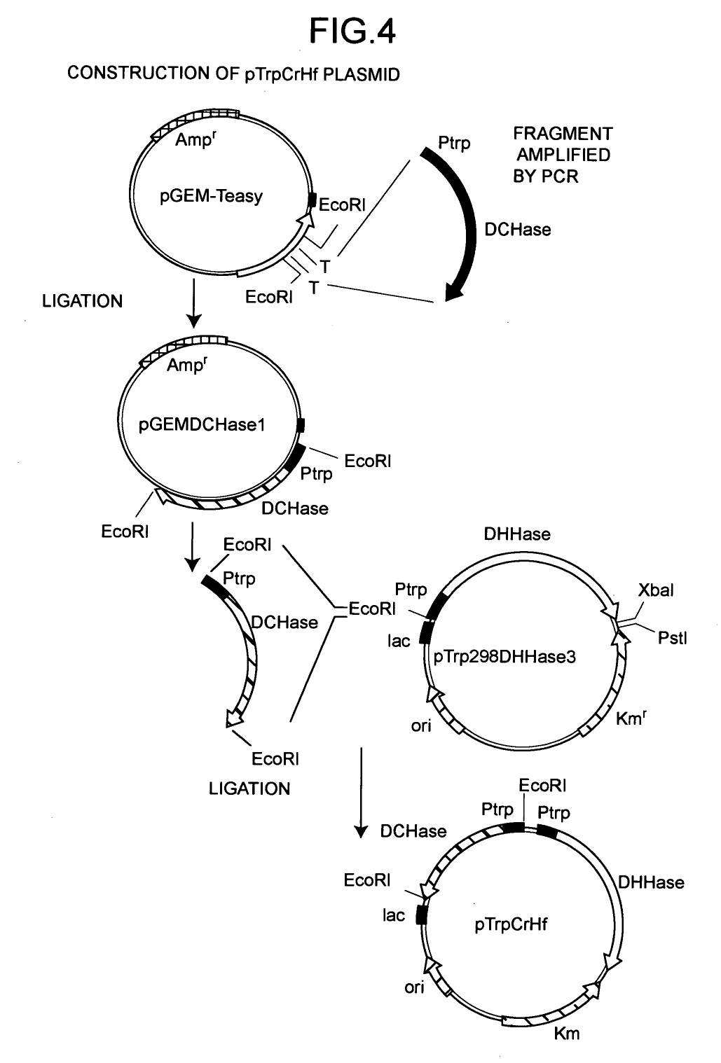

PCR was conducted with pTrp299DCHase1 as a template and the oligonucleotides (1) and (2) shown in Table 5 as primers. The resulting DNA fragment was ligated to pGEM-Teasy vector (Promega). The solution of the ligation product was used to transform E. coli JM109. Among the resulting ampicillin-resistant strains, a strain having the objective plasmid was selected, and this plasmid was designated pGEMDCHase1. Then, this plasmid was treated with EcoRI to give a DNA fragment having the trp promoter and the carbamylase gene ligated thereto, and this DNA fragment was ligated to pTrp298DHHase3 that had previously been treated with EcoRI. The solution containing the ligation product was used to transform E coli JM109. Among the resulting kanamycin-resistant strains, a strain having the objective plasmid was selected, and the plasmid was designated pTrpCrHf (FIG. 4).

1-3. Construction of pTrpHrCr plasmid

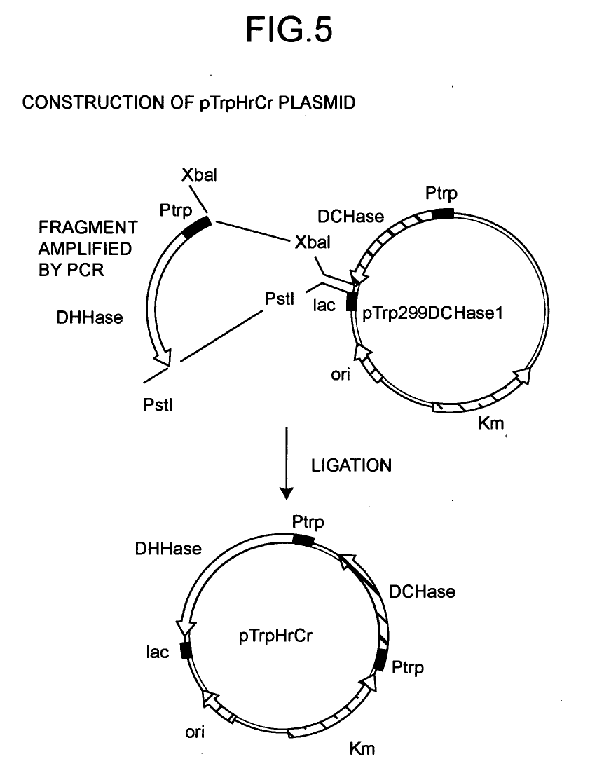

PCR was conducted with pTrp298DHHase3 as a template and the oligonucleotides (1) and (3) shown in Table 5 as primers. The resulting DNA fragment was treated with XbaI/PstI and then ligated to pTrp299DCHase1 that had previously been treated with XbaI/Pst. The solution containing the ligation product was used to transform E. coli JM109. Among the resulting kanamycin-resistant strains, a strain having the objective plasmid was selected, and this plasmid was designated pTrpHrCr (FIG. 5).

TABLE 5

Primers used in amplification of the
hydantoinase gene and carbamylase gene

| | | | |
|---|---|---|---|
| \mathring{1} | 5′-side | CGTCTAGATGTTGACAATTAATCAT | SEQ ID |
| | | XbaI | NO:18 |
| \mathring{2} | 3′-side | CGCTGCAGTCAGACGGCGGCGATCAACCCGTA | SEQ ID |
| | | PstI | NO:19 |
| \mathring{3} | 3′-side | CGCTGCAGTCAGGCCGTTTCCACTTCGCCCGT | SEQ ID |
| | | PstI | NO:20 |

2. Measurement of Enzyme Activity

Preparation of the microorganism E. coli JM109 having plasmid pTrpHfCf, pTrpCrHf, or pTrpHrCr was conducted in the same manner as the preparation performed in the measurement of enzyme activity described in Example 1. 0.2 ml of the resulting microbial suspension was added to 0.8 ml of a substrate solution (0.125 g/dl DL-benzylhydantoin, 1.25 mM MnSO₄, 100 mM KPB (pH 7.5)) and reacted at 37° C. for 60 minutes. After the reaction, 0.9 ml of the solution for terminating the reaction was added to 0.1 ml of the reaction liquid. A supernatant obtained by centrifugation was subjected to HPLC, and the amounts of formed N-carbamyl-D-Phe and D-Phe were determined. The results are shown in Table 6. In the transformant having any one of the plasmids, the co-expression of D-hydantoinase gene and D-carbamylase gene was recognized.

TABLE 6
Enzyme activity of the microorganism co-expressing D-hydantoinase
gene and D-carbamylase gene

| | D-hydantoinase activity | D-carbamylase activity |
|---|---|---|
| Plasmid | (U/ml culture liquid) | (U/ml culture liquid) |
| pTrpHfCf | 0.09 | 0.02 |
| pTrpCrHf | 0.43 | 0.17 |
| pTrpHrCr | 0.90 | 0.66 |

### Example 5

### Co-expression of D-hydantoinase gene, D-carbamylase gene and hydantoin racemase gene in E. coli

pTrp29HRase was introduced into the E. coli JM109 having pTrpHrCr to prepare a transformant having the two plasmids. This transformant was cultured at 30° C. for 16 hours in LB medium (4 ml) containing 100 μg/ml kanamycin and 50 μg/ml chloramphenicol. 1 ml of the cultured liquid thus obtained was transferred to 50 ml of medium II and cultured at 35° C. for 24 hours. After the cultivation, the microorganism was collected from 1 ml of the cultured liquid by centrifugation (10,000 g×10 minutes), and the resulting microorganism was washed with 100 mM KPB (pH 7.5). The washed microorganism was suspended in 1 ml of the same buffer, and 0.1 ml of the microbial suspension was added to 0.9 ml of a substrate solution (0.11 g/dl L-5-benzylhydantoin, 1.1 mM MnSO₄, 0.11 percent Triton X-100, 100 mM KPB (pH 7.5)) and reacted at 37° C. for 60 minutes. After the reaction, the amount of formed D-Phe was determined. It was found out that 92.7 mg/dl of D-Phe was formed, whereby co-expression of these three enzyme genes was confirmed.

### Example 6

### Production of D-Phe with the microorganism co-expressing D-hydantoinase gene, D-carbamylase gene and hydantoin racemase gene

The E. coli JM109 having two plasmids, i.e. pTrpHrCr and pTrp29HRase, was cultured at 30° C. for 16 hours in LB medium (50 ml) containing 100 μg/ml kanamycin and 50 μg/ml chloramphenicol. 15 ml of the cultured liquid thus obtained was transferred to 300 ml of medium III (1 percent casamino acid, 1 percent peptone, 2 percent glucose, 0.5 percent ammonium sulfate, 0.1 percent monopotassium phosphate, 0.3 percent dipotassium phosphate, 0.05 percent magnesium sulfate, 0.01 percent manganese sulfate, 0.01 percent kanamycin, 0.005 percent chloramphenicol (pH 7.0)) and cultured at 35° C. for 15 hours in a jar fermenter (adjusted to pH 7.0). 30 ml of the cultured liquid (300 mg as the dry weight of the microorganism) thus obtained was added to 270 ml of a substrate solution (5.5 g/dl DL-5-benzyl hydantoin, 1.11 mM MnSO₄, 0.11 percent Triton X-100, 22 mM KPB (pH 7.5)) and reacted at 37° C. for 44 hours while the pH of the reaction liquid was kept at pH 7.5 (adjusted with 1 N NaOH and 2 N H₂SO₄). As a result, it was recognized that 4.13 g/dl (molar yield, 95 percent) of D-Phe was produced.

### Example 7

### Production of D-Phe with the microorganism co-expressing

D-hydantoinase gene, D-carbamylase gene and hydantoin racemase gene (cultured in an antibiotic-free medium)

The E. coli JM109 having two plasmids, i.e. pTrpHrCr and pTrp29HRase, was cultured in kanamycin- and chloramphenicol-free LB medium (50 ml) at 30° C. for 16 hours. 15 ml of the cultured liquid thus obtained was cultured at 35° C. for 15 hours in 30 ml of kanamycin- and chloramphenicol-free medium III in a jar fermenter (adjusted to pH 7.0). 30 ml of the cultured liquid (300 mg as the dry weight of the microorganism) thus obtained was added to 270 ml of a substrate solution (5.5 g/dl DL-5-benzyl hydantoin, 1.11 mM MnSO₄, 0.11 percent Triton X-100, 22 mM KPB (pH 7.5)) and reacted at 37° C. for 44 hours while the pH of the reaction liquid was kept at pH 7.5 (adjusted with 1 N NaOH and 2 N H₂SO₄). As a result, it was recognized that 2.06 g/dl (molar yield, 48 percent) of D-Phe was produced.

### Example 8

### 1-1. Construction of a plasmid carrying trp promoter and rmB terminator

A trp operon promoter region in the chromosomal DNA of Escherichia coli W3110 was amplified as an objective gene region by PCR with the oligonucleotides shown in Table 7 as primers (combination of (1) and (2)). The resulting DNA fragment was ligated to pGEM-Teasy vector (Promega). The solution containing the ligation product was used to transform E. coli JM109. Among the resulting ampicillin-resistant strains, a strain having the objective plasmid in which the trp promoter was inserted in the reverse direction of the lac promoter was selected.

This plasmid was then treated with EcoO109I/EcoRI to obtain a DNA fragment containing the trp promoter, which was further ligated to pUC19 (Takara Shuzo) that had previously been treated with EcoO109I/EcoRI. The solution containing the ligation product was used to transform E. coli JM109. Among the resulting ampicillin-resistant strains, a strain having the objective plasmid was selected, and the plasmid was designated pTrp1.

pKK223-3 (Amersham Pharmacia) was then treated with HindIII/HincII, and the resulting DNA fragment containing rmB terminator was ligated to pTrp1 that had previously been treated with HindIII/PvuII. The solution containing the ligation product was used to transform E. coli JM109. Among the resulting ampicillin-resistant strains, a strain having the objective plasmid was selected, and the plasmid was designated pTrp2.

The trp promoter region was then amplified by PCR with pTrp2 as a template and the oligonucleotides in the table (combination of (1) and (3)) as primers. This DNA fragment was treated with EcoO109I/NdeI and then ligated to pTrp2 that had previously been treated with EcoO109/NdeI. The solution containing the ligation product was used to transform E. coli JM109. Among the resulting ampicillin-resistant strains, a strain having the objective plasmid was selected, and the plasmid was designated pTrp4.

A DNA fragment of 2.4 kb obtained by treating pSTV28 (Takara Shuzo) with EcoO109I/PvuI, a DNA fragment of 0.9 kb obtained by treating pKK223-3 (Amersham Pharmacia) with HindIII/PvuI, and a DNA fragment of 0.3 kb obtained by treating pTrp4 with EcoO109/HindIII were ligated. The solution containing the ligation product was used to transform E. coli JM109. Among the resulting chloramphenicol-resistant strains, a strain having the objective plasmid was selected, and the plasmid was designated pTrp8.

TABLE 7
Primers used in amplification of the trp promoter region

| | | | |
|---|---|---|---|
| \mathring{1} | 5′-side | GTATCACGAGGCCCTAGCTGTGGTGTCATGGTCGGTGATC | SEQ ID NO:23 |
| | | Eco0109I | |
| \mathring{2} | 3′-side | TTCGGGGATTCCATATGATACCCTTTTTACGTGAACTTGC | SEQ ID NO:24 |
| | | NdeI | |
| \mathring{3} | 3′-side | GGGGGGGGCATATGCGACCTCCTTATTACGTGAACTTG | SEQ ID NO:25 |
| | | NdeI | |

1-2. Construction of a plasmid carrying a hydantoin racemase gene

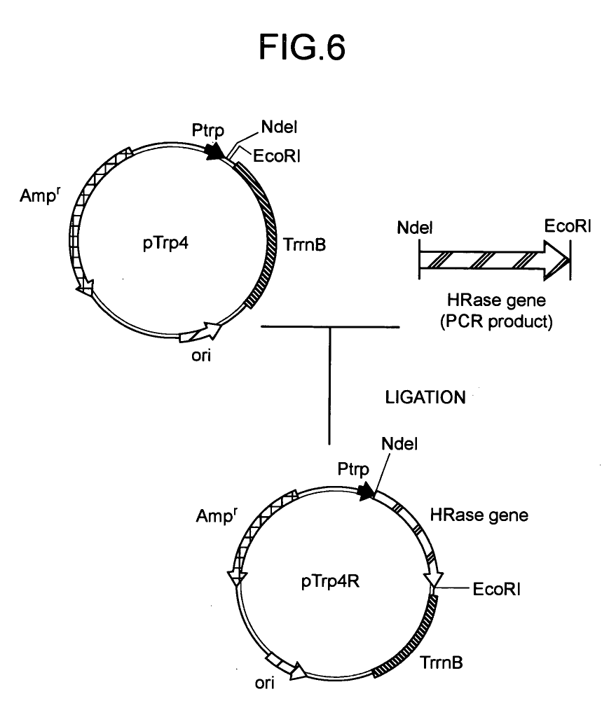

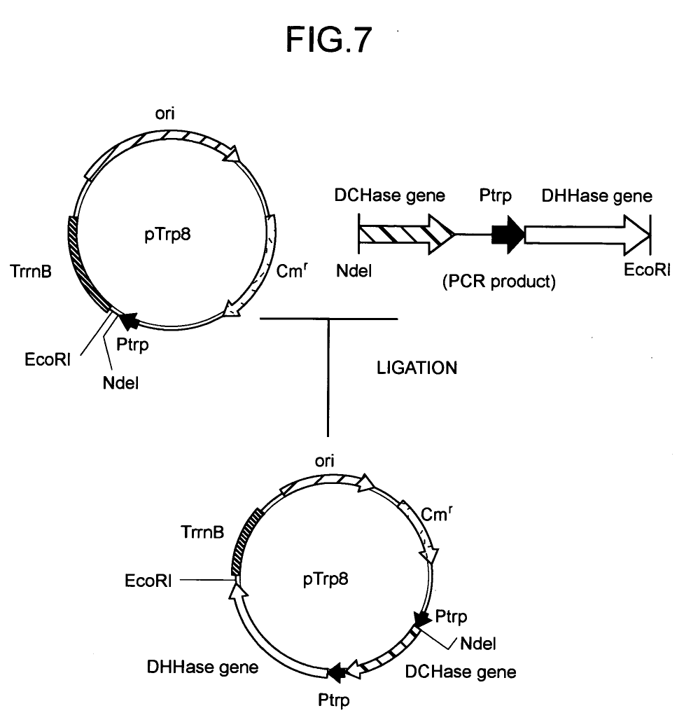

The objective gene was amplified by PCR with a chromosomal DNA from Microbacterium liquefaciens AJ 3912 as a template and the oligonucleotides shown in Table 8 as primers. This fragment was treated with NdeI/EcoRI, and the resulting DNA fragment was ligated to pTrp4 that had previously been treated with NdeI/EcoRI. The solution containing the ligation product was used to transform E. coli JM109. Among the resulting ampicillin-resistant strains, a strain having the objective plasmid was selected, and the plasmid was designated pTrp4R (FIG. 6). In FIGS. 6 and 7, the rmB terminator is referred to as “TrmB”.

TABLE 8

Primers used in amplification of the
hydantoin racemase gene derived from AJ3912

| | | | |
|---|---|---|---|
| \mathring{1} | 5′-side | CGGGAATTCCATATGCGTATCCATGTCATCAA | SEQ ID |
| | | NdeI | NO:26 |
| \mathring{2} | 3′-side | CGCGGATCCTTAGAGGTACTGCTTCTCGG | SEQ ID |
| | | EcoRI | NO:27 |

1-3. Construction of a plasmid carrying a D-hydantoinase gene and D-carbamylase gene

The objective genes were amplified by PCR with pTrpHrCr as a template and the oligonucleotides shown in Table 9 as primers. This fragment was treated with NdeI/EcoRI, and the resulting DNA fragment was ligated to pTrp8 that had previously been treated with NdeI/EcoRI. The solution containing the ligation product was used to transform E. coli JM109. Among the resulting chloramphenicol-resistant strains, a strain having the objective plasmid was selected, and the plasmid was designated pTrp8CH (FIG. 7).

TABLE 9

Primers used in amplification of the
D-hydantoinase and D-carbamylase gene

| | | | |
|---|---|---|---|
| \mathring{1} | 5′-side | GGAATTCCATATGCCAGGAAAGATCATTCTCGCG | SEQ ID NO:28 |
| | | NdeI | |
| \mathring{2} | 3′-side | CGGAATTCTCAGGCCGTTTCCACTTCGCC | SEQ ID NO:29 |
| | | EcoRI | |

1-4. Preparation of a strain co-expressing the D-hydantoinase gene, D-carbamylase gene and hydantoin racemase gene, and production of D-Phe

The E. coli JM109 that already had pTrp4R was transformed by introducing pTrp8CH thereinto, to prepare a transformant having these two plasmids. This transformant was cultured at 30° C. for 16 hours in LB medium (50 ml) containing 100 μg/ml ampicillin and 50 μg/ml chloramphenicol. 1 ml of the cultured liquid thus obtained was transferred to 300 ml of medium IV (2.5 percent glucose, 0.5 percent ammonium sulfate, 0.14 percent monopotassium dihydrogen phosphate, 0.23 percent trisodium citrate-dihydrate, 0.002 percent iron(II) sulfate-heptahydrate, 0.1 percent magnesium sulfate-heptahydrate, 0.002 percent manganese sulfate-pentahydrate, 0.0001 percent thiamine hydrochloride, 0.01 percent ampicillin, 0.005 percent chloramphenicol (pH 7.0)), and cultured at 33° C. for 24 hours in a jar fermenter while the pH was regulated at 7.0 and the dissolved oxygen concentration was regulated at 1.5 ppm or more. 15 ml of the cultured liquid thus obtained was transferred to 300 ml of medium V (2.5 percent glucose, 0.5 percent ammonium sulfate, 0.3 percent phosphoric acid, 0.23 percent trisodium citrate-dihydrate, 0.1 percent magnesium sulfate-heptahydrate, 0.002 percent iron(II) sulfate-heptahydrate, 0.002 percent manganese sulfate-pentahydrate, 0.0001 percent thiamine hydrochloride, 0.01 percent ampicillin, 0.005 percent chloramphenicol (pH 7.0)) and cultured at 35° C. for 24 hours in a jar fermenter while the pH was regulated at 7.0, and the dissolved oxygen concentration was regulated at 1.5 ppm or more. After the lapse of 9 hours from the beginning of the cultivation, 50 percent aqueous glucose solution was added thereto at the addition rate of 3.5 ml/hr. 15 ml of the cultured liquid (600 mg as the dry weight of the microorganism) thus obtained was added to 285 ml of a substrate solution (5.3 g/dl DL-5-benzyl hydantoin, 1.05 mM manganese sulfate, 21 mM KPB (pH 7.5)) and reacted at 37° C. for 48 hours while the pH of the reaction liquid was kept at pH 7.5 (adjusted with 1 N NaOH and 2 N H₂SO₄). As a result, it was recognized that 4.15 g/dl (molar yield, 95 percent) of D-Phe was produced.

1-5. Production of D-Phe with the strain co-expressing the D-hydantoinase gene, D-carbamylase gene and hydantoin racemase gene (antibiotic-free system)

The E. coli JM109 having the two plasmids, i.e. pTrp4R and pTrp8CH, was cultured at 30° C. for 16 hours in LM medium (50 ml) that was free of ampicillin and chloramphenicol. 1 ml of the cultured liquid thus obtained was transferred to 300 ml of medium IV that was free of ampicillin and chloramphenicol and cultured at 33° C. for 24 hours in ajar fermenter while the pH was regulated at 7.0 and the dissolved oxygen concentration was regulated at 1.5 ppm or more. 15 ml of the cultured liquid thus obtained was transferred to 300 ml of medium V that was free of ampicillin and chloramphenicol and cultured at 35° C. for 24 hours in a jar fermenter while the pH was regulated at 7.0 and the dissolved oxygen concentration was regulated at 1.5 ppm or more. After the lapse of 9 hours from the beginning of the cultivation, 50 percent aqueous glucose solution was added thereto at the addition rate of 3.5 ml/hr. 15 ml of the cultured liquid (600 mg as the dry weight of the microorganism) thus obtained was added to 285 ml of a substrate solution (5.3 g/dl DL-5-benzylhydantoin, 1.05 mM manganese sulfate, 21 mM KPB (pH 7.5)) and reacted at 37° C. for 48 hours while the pH of the reaction liquid was kept at pH 7.5 (adjusted with 1 N NaOH and 2 N H₂SO₄). As a result, it was found that this antibiotic-free cultivation did not result in disappearance of the plasmids, and that 4.15 g/dl (molar yield, 95 percent) of D-Phe was produced.

### INDUSTRIAL APPLICABILITY

According to the present invention, there is provided a recombinant DNA co-expressing a hydantoinase gene and a carbamylase gene efficiently. According to the present invention, amino acids can be produced with high productivity.

| | | Dates of original | |
|---|---|---|---|
| | | deposition and transfer | |
| | Accession | to the international | Name and address of the |
| Name | Number | deposition | depositary authority |
| Agrobacterium | FERM | Date of original | Name: International Patent |
| sp. AJ 11220 | BP-7645 | deposition: | Organism Depositary, |
| | | Dec. 20, 1977 | National Institute of |
| | | Date of transfer to the | Advanced Industrial |
| | | international deposition: | Science and Technology |
| | | Jun. 27, 2001 | Address: Tsukuba Central |
| | | | 6, 1-1-1 Higashi, Tsukuba, |
| | | | Ibaraki, Japan |
| Microbacterium | FERM | Date of original | Name: International Patent |
| liquefaciens AJ 3912 | BP-7643 | deposition: | Organism Depositary |
| | | Jun. 27, 1975 | National Institute of |
| | | Date of transfer to the | Advanced Industrial |
| | | international deposition: | Science and Technology |
| | | Jun. 27, 2001 | Address: Tsukuba Central |
| | | | 6, 1-1-1 Higashi, Tsukuba, |
| | | | Ibaraki, Japan |
| Flavobacterium | FERM | Date of original | Name: International Patent |
| sp. AJ 11199 | BP-8063 | deposition: May 1, 1981 | Organism Depositary, |
| | | Date of transfer to the | National Institute of |
| | | international deposition: | Advanced Industrial |
| | | May 30, 2002 | Science and Technology |
| | | | Address: Tsukuba Central |
| | | | 6, 1-1-1 Higashi, Tsukuba, |
| | | | Ibaraki, Japan |

1. A recombinant DNA comprising the following gene regions (I) and (II):
(I) a gene region containing a nucleotide sequence encoding a hydantoinase gene, and a trp promoter sequence located upstream of said nucleotide sequence encoding said hydantoinase gene, said trp promoter regulating expression of said hydantoinase gene;
(II) a gene region containing a nucleotide sequence encoding a carbamylase gene, and a trp promoter sequence located upstream of said nucleotide sequence encoding said carbamylase gene, said trp promoter regulating expression of said carbamylase gene.

2. The recombinant DNA according to claim 1, wherein the transcriptional direction of said gene region (I) is the same as that of said gene region (II), and wherein said gene region (I) and said gene region (II) are disposed in this order along said transcriptional direction.

3. The recombinant DNA according to claim 1, wherein the transcriptional direction of said gene region (I) is in the reverse direction of said gene region (II).

4. The recombinant DNA according to claim 1, wherein the transcriptional direction of said gene region (I) is the same as that of said gene region (II), and wherein said gene region (II) and said gene region (I) are disposed in this order along said transcriptional direction.

5. The recombinant DNA according to claim 1, wherein said recombinant DNA is a plasmid.

6. A transformed cell comprising said recombinant DNA according to claim 1.

7. The transformed cell according to claim 6 further comprising a recombinant DNA having a nucleotide sequence encoding a hydantoin racemase gene and a trp promoter sequence located upstream of said nucleotide sequence encoding said hydantoin racemase gene, said trp promoter regulating expression of said hydantoin racemase gene.

8. The transformed cell according to claim 7, wherein said recombinant DNA containing said gene regions (I) and (II) is a low-copy recombinant DNA, and said recombinant DNA having said nucleotide sequence encoding said hydantoin racemase gene and said trp promoter sequence located upstream of said hydantoin racemase gene regulating expression of said hydantoin racemase gene is a high-copy recombinant DNA.

9. The transformed cell according to claim 6, wherein the host of said recombinant DNA is an Escherichia coli bacterium.

10. A process for producing a mixture of proteins comprising
culturing the transformed cell according to claim 6 in a medium, to accumulate a protein having a hydantoinase activity and a protein having a carbamylase activity in said medium and/or said transformed cell for a time and under conditions suitable for expression of said proteins to produce a mixture of proteins, and
collecting said mixture of proteins.

11. A process for producing a mixture of proteins comprising
culturing said transformed cell according to claim 7 in a medium, to accumulate a protein having a hydantoinase activity, a protein having a carbamylase activity and a protein having a hydantoin racemase activity in said medium and/or said transformed cell for a time and under conditions suitable for expression of said proteins to produce a mixture of proteins and
collecting said mixture of proteins.

12. A process for producing an amino acid comprising
culturing the transformed cell according to claim 6 in a medium to give a culture, and
contacting 5-substituted hydantoin with said culture to form said amino acid.

13. A process for producing an amino acid comprising
culturing the transformed cell according to claim 7 in a medium to give a culture, and
contacting 5-substituted hydantoin with said culture to form said amino acid.

14. The recombinant DNA according to claim 1, wherein said nucleotide sequence encoding a hydantoinase gene is selected from the group consisting of:
(i) a DNA having the nucleotide sequence of SEQ ID NO:1,
(ii) a DNA having a nucleotide sequence that hybridizes under stringent conditions with a DNA consisting of a nucleotide sequence complementary to the nucleotide sequence of SEQ ID NO: 1,
(iii) a DNA encoding an amino acid sequence of SEQ ID NO:2, and
(iv) a DNA encoding an amino acid sequence wherein in the amino acid sequence of SEQ ID NO:2, one or several amino acid residues are replaced, deleted, inserted, added or inverted and said amino acid sequence has hydantoinase activity.

15. The recombinant DNA according to claim 1, wherein said nucleotide sequence encoding a carbamylase gene is selected from the group consisting of:
(i) a DNA having the nucleotide sequence of SEQ ID NO:3,
(ii) a DNA having a nucleotide sequence that hybridizes under stringent conditions with a DNA consisting of a nucleotide sequence complementary to the nucleotide sequence of SEQ ID NO:3,
(iii) a DNA encoding an amino acid sequence of SEQ ID NO:4, and
(iv) a DNA encoding an amino acid sequence wherein in the amino acid sequence of SEQ ID NO:5, one or several amino acid residues are replaced, deleted, inserted, added or inverted and said amino acid sequence has carbamylase activity.

16. The transformed cell according to claim 6, wherein the transcriptional direction of said gene region (I) is the same as that of said gene region (II), and wherein said gene region (II) and said gene region (I) are disposed in this order along said transcriptional direction.

17. The transformed cell according to claim 7, wherein said nucleotide sequence encoding a hydantoin racemase gene is selected from the group consisting of:
(i) a DNA having the nucleotide sequence of SEQ ID NO:5,
(ii) a DNA having a nucleotide sequence that hybridizes under stringent conditions with a DNA consisting of a nucleotide sequence complementary to the nucleotide sequence of SEQ ID NO:5,
(iii) a DNA encoding an amino acid sequence of SEQ ID NO:6, and
(iv) a DNA encoding an amino acid sequence wherein in the amino acid sequence of SEQ ID NO:6, one or several amino acid residues are replaced, deleted, inserted, added or inverted and said amino acid sequence has hydantoin racemase activity.

18. The process according to claim 12, wherein said 5-substituted hydantoin is selected from the group consisting of hydantoin, 5-methyl hydantoin, 5-benzyl hydantoin, 5-(4-hydroxybenzyl)hydantoin, 5-indolylmethyl hydantoin, 5-(3,4-dihydroxybenzyl)hydantoin, 5-(p-hydroxybenzyl)hydantoin, 5-(3′-pyridyl)-methyl hydantoin, 5-methyl thioethyl hydantoin, 5-isopropyl hydantoin, 5-isobutyl hydantoin, 5-sec-butyl hydantoin, 5-carboxyethyl hydantoin, 5-carboxymethyl hydantoin, 5-(4-aminobutyl)hydantoin, and 5-hydroxymethyl hydantoin, as well as 5-substituted hydantoins corresponding to artificial amino acids such as 5-phenyl hydantoin, 5-(4-hydroxyphenyl)hydantoin, 5-methoxymethyl hydantoin, 5-benzyloxymethyl hydantoin, and 5-(3,4-methylenedioxybenzyl)hydantoin, and dihydrouracil, or derivatives thereof.

19. The process according to claim 13, wherein said 5-substituted hydantoin is selected from the group consisting of hydantoin, 5-methyl hydantoin, 5-benzyl hydantoin, 5-(4-hydroxybenzyl)hydantoin, 5-indolylmethyl hydantoin, 5-(3,4-dihydroxybenzyl)hydantoin, 5-(p-hydroxybenzyl)hydantoin, 5-(3′-pyridyl)-methyl hydantoin, 5-methyl thioethyl hydantoin, 5-isopropyl hydantoin, 5-isobutyl hydantoin, 5-sec-butyl hydantoin, 5-carboxyethyl hydantoin, 5-carboxymethyl hydantoin, 5-(4-aminobutyl)hydantoin, and 5-hydroxymethyl hydantoin, as well as 5-substituted hydantoins corresponding to artificial amino acids such as 5-phenyl hydantoin, 5-(4-hydroxyphenyl)hydantoin, 5-methoxymethyl hydantoin, 5-benzyloxymethyl hydantoin, and 5-(3,4-methylenedioxybenzyl)hydantoin, and dihydrouracil, or derivatives thereof.


SEQUENCE LISTING (31 sequences)

SEQ ID NO: 1 - DNA, 1458 bp, Flavobacterium sp
  CDS (1)..(1455)
atgacccattacgatctcgtcattcgcggaggaaccgtcgccacggcgagtgattgcttccgggcggatgttgcggtcaccgacggcaggatcgtcgccatcggcgagaatctcgggcccgccgcccgcgagatctcggcggaaggccgcctcgttctgcccggcggcgtcgattcccattgccatatcgaggagccgcaggtcggcgaggtgcgcaacgcggaaaccttcgcctccgccacctcttcggcggccgcgggcggcacgacgacggtcatttccttctctcagcaggtgaaaggcggcggcatcaccgaggccctgcgcgactatcacgagaaggcgacccgcgcgctgatcgactattccttccatctcgtcgtgaccgatcccacggatgcggtgctggaggaactggtgccgctgatcgaggaagggcaccggtcgctgaagatcttcatgacctataccaatgtcgtgctcgacgacgagcagacgctgcgcgtgctggcgctcgcccgcaagaccggcgctctcgtcacggtgcatgccgagaaccatgccgcgatcatgtacctcacgcgtgccttggaaaaggccgggctcaccgccccgaagtaccacgcctgggccaagcccatgccggtggaacgggaggcctgccaccgcatcatcatgctctccgaactgctggatgttcccattcagatcttccacgtttccggggcggaggcggccgaggaaatccgccgcgcgcagaatcgcggcctcaaggtcttcggcgagacctgcccgcaatatctgatgctgacggcggcggatctcgaccggccggatttcgagggtgcgaaattcctgtgcagccccgcgccgcgcacgacggccgatcaggaagccctgtgggactatatccgcacgggaacgatcggggtcgtgtcgtccgaccacgcgccgaaccgcttcgacgatccgcacggcaagaaggtcggcggggtggacgcgcccttcagcgccattccgaacggcgtgccggggctggcgacgcgcctgccgatcctgttttccgaaggggtcgtcaaggggcgcatcgacctcaacaccttcgtcgcgctgacggcggccaatccggcgaagctctttggcctgcatcccagaaagggcaccatcgccgtcggagccgatgcggatatcgcgatctgggatcccgagcgcaaggtgacgatccgcaacgacatgctgcatcacggcgtcgactacacggtctacgagggcgtcgaggtgaccggatggccggtgatgacgctctcgcgcggcgaggtggtctatgatgacggcgcggtggtcggcaagccgggtcacgggcgcttccttgcccgcggtccctatgacgcgatcaagccgcgcggcaggcatgtgacgcctttcgacccggtgacgggcgaagtggaaacggcctga

SEQ ID NO: 2 - protein, 485 aa, Flavobacterium sp
MTHYDLVIRGGTVATASDCFRADVAVTDGRIVAIGENLGPAAREISAEGRLVLPGGVDSHCHIEEPQVGEVRNAETFASATSSAAAGGTTTVISFSQQVKGGGITEALRDYHEKATRALIDYSFHLVVTDPTDAVLEELVPLIEEGHRSLKIFMTYTNVVLDDEQTLRVLALARKTGALVTVHAENHAAIMYLTRALEKAGLTAPKYHAWAKPMPVEREACHRIIMLSELLDVPIQIFHVSGAEAAEEIRRAQNRGLKVFGETCPQYLMLTAADLDRPDFEGAKFLCSPAPRTTADQEALWDYIRTGTIGVVSSDHAPNRFDDPHGKKVGGVDAPFSAIPNGVPGLATRLPILFSEGVVKGRIDLNTFVALTAANPAKLFGLHPRKGTIAVGADADIAIWDPERKVTIRNDMLHHGVDYTVYEGVEVTGWPVMTLSRGEVVYDDGAVVGKPGHGRFLARGPYDAIKPRGRHVTPFDPVTGEVETA

SEQ ID NO: 3 - DNA, 915 bp, Flavobacterium sp
  CDS (1)..(912)
atgccaggaaagatcattctcgcggtgggccagcttggacccatcgcccgcagcgacagccgcgaacaggtggtgaagcggctcgtcgacatgctggaggaggccgcggcccgcaattcggatttcatcgtctttcccgagctggcgctgaccaccttcttcccccgctggtatttcaccgacgaggcggaactggacgcgttctacgagacggaaatgccgagcccggtgacacgcccgctgttcgaggccgccgcgaaacacggcgtcgggttcaatctcggcttcgcggagctcgtgtcggagggcggcaggaagcgccgcttcaacacgtccatcgccgtcgacaggaccggcaggatcatcggcaagtaccgcaagatccatctgccgggccacaaggactatgaagactaccgcccgttccagcatctggagaagcggtacttcgagaccggcaatctcggattcccggtctatgaggtcgatggtttcaagatgggcatgttcatctgcaacgaccggcgctggccggagacctggcgcgtgatgggtctcaagggcgcggagctgatctgcggcggctacaacacgccgctgcacaatccgccggtgccgcagcacgaccagctcagctccttccaccatctcctgtcgatgcaggcgggcgcctaccagaacggtgcgtggaccgccggagcgggcaaggtcggggtggaggaaggctgcatgctgctcggccattcctgcatcgtcgcgcccaccggggagcttgccgcgctcaccaacacgctggaggacgaggtcatcacggccgctgccgattttgcccgctgccgggaaatccgcgagaacatcttcaatttcgagcagcatcgcgagccgcaggaatacgggttgatcgccgccgtctga

SEQ ID NO: 4 - protein, 304 aa, Flavobacterium sp
MPGKIILAVGQLGPIARSDSREQVVKRLVDMLEEAAARNSDFIVFPELALTTFFPRWYFTDEAELDAFYETEMPSPVTRPLFEAAAKHGVGFNLGFAELVSEGGRKRRFNTSIAVDRTGRIIGKYRKIHLPGHKDYEDYRPFQHLEKRYFETGNLGFPVYEVDGFKMGMFICNDRRWPETWRVMGLKGAELICGGYNTPLHNPPVPQHDQLSSFHHLLSMQAGAYQNGAWTAGAGKVGVEEGCMLLGHSCIVAPTGELAALTNTLEDEVITAAADFARCREIRENIFNFEQHREPQEYGLIAAV

SEQ ID NO: 5 - DNA, 711 bp, Microbacterium liquefaciens
  CDS (1)..(708)
atgagaatccatgtcatcaatcccaacagctcggtggatctcaccgatgcggtggccgaggcggcgcgaagtgtggtgtcaccgggaaccaccatcaccgcggtcaacccttcgaagggccccacggtcatcgagggcagttacgacgaggtgctggccacgtatcacctcgtcgaagaggtccgccgcgcggagcgcgaagaccgaccggacgcctacgtcatcgcctgtttcggcgatccaggtctcgacgccgtcagggagctcaccgacaggcccgtggtcggaatcgccgaggcggcgatccagatgacgagcttcgtcgccgcgagcttctccatcgtgagcatcctcccgcgcgtgcgcaagcatctgcacgagctggtgcaccgggcgggggcaacggatcgactcgcctcactcaagcttccggatctcggagtgctcgcattccacgaggacgaggcagcggcgttcgagaccctccggcgcgtggcaggtgaggcggtgcgcgaggacggcgcggagtcgatcgtgctcggctgcgcgggcatggccggattcgccagacagctgagtgaagagctcggcgtccccgtcatcgacgcggtcgaggcagcctgccgcgtcgcggagagcctcgtcgccctggggtaccgcaccagcaaggcgaacacctaccaagcacccaccgagaagcagtacctctaa

SEQ ID NO: 6 - protein, 236 aa, Microbacterium liquefaciens
MRIHVINPNSSVDLTDAVAEAARSVVSPGTTITAVNPSKGPTVIEGSYDEVLATYHLVEEVRRAEREDRPDAYVIACFGDPGLDAVRELTDRPVVGIAEAAIQMTSFVAASFSIVSILPRVRKHLHELVHRAGATDRLASLKLPDLGVLAFHEDEAAAFETLRRVAGEAVREDGAESIVLGCAGMAGFARQLSEELGVPVIDAVEAACRVAESLVALGYRTSKANTYQAPTEKQYL

SEQ ID NO: 7 - DNA, 26 bp, Artificial Sequence
  Synthetic DNA
ggggtaccggagctgataccatgacc

SEQ ID NO: 8 - DNA, 26 bp, Artificial Sequence
  Synthetic DNA
ggggtaccgcaaccgatagccggagc

SEQ ID NO: 9 - DNA, 26 bp, Artificial Sequence
  Synthetic DNA
ggggtaccatgacccattacgatctc

SEQ ID NO: 10 - DNA, 26 bp, Artificial Sequence
  Synthetic DNA
ggggtaccaccatgacccattacgat

SEQ ID NO: 11 - DNA, 26 bp, Artificial Sequence
  Synthetic DNA
gctctagacgtcctgtcctttccgcc

SEQ ID NO: 12 - DNA, 26 bp, Artificial Sequence
  Synthetic DNA
ggggtaccggagaggaatatgccagg

SEQ ID NO: 13 - DNA, 27 bp, Artificial Sequence
  Synthetic DNA
ggggtaccgggaaaggaaaaggagagg

SEQ ID NO: 14 - DNA, 26 bp, Artificial Sequence
  Synthetic DNA
gctctagagccgcgccgctcagacgg

SEQ ID NO: 15 - DNA, 26 bp, Artificial Sequence
  Synthetic DNA
gctctagaatgagaatccatgtcatc

SEQ ID NO: 16 - DNA, 26 bp, Artificial Sequence
  Synthetic DNA
gctctagagcgatgcgtatccatgtc

SEQ ID NO: 17 - DNA, 26 bp, Artificial Sequence
  Synthetic DNA
ggctgcagcgctccttctcgttagag

SEQ ID NO: 18 - DNA, 25 bp, Artificial Sequence
  Synthetic DNA
cgtctagatgttgacaattaatcat

SEQ ID NO: 19 - DNA, 32 bp, Artificial Sequence
  Synthetic DNA
cgctgcagtcagacggcggcgatcaacccgta

SEQ ID NO: 20 - DNA, 32 bp, Artificial Sequence
  Synthetic DNA
cgctgcagtcaggccgtttccacttcgcccgt

SEQ ID NO: 21 - DNA, 67 bp, Artificial Sequence
  Synthetic DNA
aattctgttgacaataattcatcgaactagttaactagtacgcaagttcacgtaaaaagggtggtac

SEQ ID NO: 22 - DNA, 63 bp, Artificial Sequence
  Synthetic DNA
aattctgttgacaataattcatcgaactagttaactagtacgcaagttcacgtaaaaagggtt

SEQ ID NO: 23 - DNA, 40 bp, Artificial Sequence
  Synthetic DNA
gtatcacgaggccctagctgtggtgtcatggtcggtgatc

SEQ ID NO: 24 - DNA, 40 bp, Artificial Sequence
  Synthetic DNA
ttcggggattccatatgataccctttttacgtgaacttgc

SEQ ID NO: 25 - DNA, 38 bp, Artificial Sequence
  Synthetic DNA
ggggggggcatatgcgacctccttattacgtgaacttg

SEQ ID NO: 26 - DNA, 32 bp, Artificial Sequence
  Synthetic DNA
cgggaattccatatgcgtatccatgtcatcaa

SEQ ID NO: 27 - DNA, 29 bp, Artificial Sequence
  Synthetic DNA
cgcggatccttagaggtactgcttctcgg

SEQ ID NO: 28 - DNA, 34 bp, Artificial Sequence
  Synthetic DNA
ggaattccatatgccaggaaagatcattctcgcg

SEQ ID NO: 29 - DNA, 29 bp, Artificial Sequence
  Synthetic DNA
cggaattctcaggccgtttccacttcgcc

SEQ ID NO: 30 - DNA, 59 bp, Artificial Sequence
  Synthetic DNA
caccctttttacgtgaacttgcgtactagttaactagttcgatgattaattgtcaacag

SEQ ID NO: 31 - DNA, 63 bp, Artificial Sequence
  Synthetic DNA
ctagaaccctttttacgtgaacttgcgtactagttaactagttcgatgattaattgtcaacag